(19) United States
(12) Patent Application Publication
(10) Pub. No.: US 2022/0192994 A1
(43) Pub. Date: Jun. 23, 2022

(54) SOFTGEL FILL MATERIAL

(71) Applicant: BARLEAN'S ORGANIC OILS, LLC, Ferndale, WA (US)

(72) Inventor: Peter Nguyen, Renton, WA (US)

(73) Assignee: BARLEAN'S ORGANIC OILS, LLC, Ferndale, WA (US)

(21) Appl. No.: 17/653,697

(22) Filed: Mar. 7, 2022

Related U.S. Application Data
(63) Continuation of application No. PCT/US2020/050160, filed on Oct. 9, 2020.
(60) Provisional application No. 62/899,017, filed on Sep. 11, 2019.
(60) Provisional application No. 62/946,306, filed on Dec. 10, 2019.

(51) Int. Cl. A61K 9/48; A61K 31/05; A61K 35/60; A61K 36/55; A61K 36/71

(52) U.S. Cl. CPC A61K 9/4858; A61K 31/05; A61K 36/71; A61K 36/55; A61K 35/60

(57) ABSTRACT

Provided is a semisolid softgel fill material that includes water and/or other polar solvents, along with a high amount of a nonpolar component, such as an oil. Also provided are methods and systems for producing softgels including the fill material. The softgels produced by the methods and systems provided herein have a normal appearance and retain their structural integrity, even when dried according to conventional methods. Further, when the fill material is added to water or other liquid, as it would be following ingestion, the fill material forms small droplets, thereby enhancing digestion and absorption of the fill material.

### CROSS-REFERENCE TO RELATED APPLICATIONS

This application is a continuation of and claims priority benefit to International Patent Application No. PCT/US20/50160 (WO2021/050697), filed Sep. 10, 2020, and titled “Softgel Fill Materials,” which claims the benefit of and priority to U.S. Provisional Application No. 62/899,017, filed Sep. 11, 2019, titled “Softgel Fill Material” and to U.S. Provisional Application No. 62/946,306, filed Dec. 10, 2019, also titled “Softgel Fill Material,” all of which are expressly incorporated herein by reference in their entirety.

### TECHNICAL FIELD

The present disclosure relates generally to softgel fill materials, and more particularly to softgel fill material compositions that can be quickly absorbed when ingested and that can be used to make softgels that are easily dried by conventional methods. Also provided are methods and systems for making the fill materials, along with methods and systems for making softgels including the fill material.

### BACKGROUND

A conventional gelatin capsule or “softgel” is a one-piece, hermetically sealed soft gelatin shell containing a liquid, a suspension, or other fill material. In the nutraceutical industry, for example, softgels frequently contain oils, which are believed to provide several health benefits. When ingested, the oily fill materials of such softgels are slowly broken down in the intestine and eventually absorbed by the user, along with any beneficial components that are included within the oil. Unlike other food components, such as proteins and sugars, oils generally take longer to digest, with the amount of time varying based on the type of oil.

To make a conventional oil-filled softgel, manufactures typically rely on a rotary die process. For example, a heated mixture of gelatin, water, glycerol and other components are used to form two flexible gelatin sheets or ribbons. The ribbons are then synchronously guided between two mated dies. A pump simultaneously delivers the oil-based fill material into a heated wedge that sits between the rotary dies. The pump injects fill material into the die cavities between the ribbons just before the die rolls cut the ribbons and seal the two cut halves of the ribbon together to form a softgel. The warm, newly formed softgels are then collected as they exit the dies and are dried. The dried softgels are then packaged for shipment to the customer.

Because the composition of the softgel walls and the fill material can be different, great care must be taken, however, when drying softgels so that they retain their structural integrity and appearance well after they are packaged and sold to consumers. To expedite the drying process of oil-based softgel fills, softgels can be dried using drying tunnels in which the softgels are placed on trays and slowly dried over several days. In other, more recent drying techniques, softgels with oil-based fills can be dried much faster using a series of drying zones and tumble driers.

While the use of tunnel drying and/or drying-zone tumble dryers work well for softgels having oil-based fills, such as fish oils, drying softgels with water or other polar components in the fill material remains highly problematic. For example, having even small amounts of water in the fill material during the drying process can lead to misshapen and uncured softgels because the water can migrate from the fill material to the softgel walls and into the environment. This in turn can adversely affect the shape and structural integrity of the softgel.

Hence, what is needed are softgels that can be dried using conventional drying means but that also include water or other polar solvents as part of the fill material. More particularly, what is needed are softgels and softgel fill materials that include oil and water in the fill material, for example, but that can be dried using conventional drying means without shriveling, becoming too soft or mushy, or failing to fully form and cure during the drying process. Also needed are softgel fill materials that can include water in addition to oil in the fill material, thereby providing a fill material that is more readily digested and absorbed as compared to an oil-filled softgel.

### SUMMARY

In certain example aspects, provided is a softgel including a semisolid fill material. The semisolid fill material of the softgel includes, for example, a first lipid component and a second lipid component, with the first lipid component and the second lipid component being a total of at least 60% by weight of the fill material. The semisolid fill material of the softgel also includes a surfactant, such as d-α-tocopheryl polyethylene glycol succinate (“TPGS”) or a derivative thereof, along with water and/or other polar solvent, with the total amount of the water and/or other polar solvent being about 10% by weight or less of the fill material. In certain example aspects, the water and/or other polar solvent about 5% by weight or less of the semisolid fill material of the softgel. In certain example aspects, the TPGS or derivative thereof is present at about 8-15% by weight of the fill material, such as about 13% by weight of the fill material.

In certain example aspects, the semisolid fill material of the softgel also includes an emulsifier, such as gum Arabic, starch, or a combination thereof. In certain example aspects, the fill material includes less than about 5% by weight of the emulsifier.

In certain example aspects, the first lipid component of the semisolid fill material of the softgel is a waxy oil or mixture of waxy oils while the second lipid component of the semisolid fill material is a non-waxy oil or mixture of non-waxy oils. The waxy oil (or mixture thereof) and non-waxy oil (or mixture thereof) are present in a predetermined ratio, such as a ratio of at least 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1 or 30:1 (waxy oil to non-waxy oil). In certain example aspects, the ratio is about 12:1 or about 25:1. In certain example aspects, the first lipid component and the second lipid component include a total of at least 65% to 70% by weight of the fill material of the softgel.

In certain example aspects, the semisolid fill material of the softgel includes other components, such as coconut oil, the coconut oil being at least 40%, 50%, or 60% by weight of the semisolid fill material of the softgel. In certain example aspects, the semisolid fill material of the softgel includes beeswax, with the beeswax being about 4% or more by weight of the fill material. Other components of the semisolid fill material of the softgel can include for example, lecithin (such as at 4-6% by weight of the fill material) and glycerin (such as around 3-6% by weight of the fill material).

In certain example aspects, the semisolid fill material of the softgel includes an active ingredient. For example, the active ingredient can be a nonpolar active ingredient, with the nonpolar active ingredient being included as part of the first and/or second lipid component. In certain example aspects, the active ingredient is a cannabidiol (CBD), a fish oil, a flax seed oil, a black seed oil, or combination thereof.

In certain example aspects, the first and second lipid components of the semisolid fill material of the softgel are dispersed within the water and/or other polar solvent of the fill material. As such, in certain example aspects the lipid components form droplets within the dispersion, the droplets having a median or average particle size of about 15 to 20 μm. In certain example aspects, at least 85% of the droplets have a particle size of at least 40-50 μm. In certain example aspects, the droplets have a D50 of about 15-20 μm and/or a D90 of about 45 μm.

In further example aspects, provided is a semisolid softgel fill material. The semisolid fill material includes, for example, a waxy oil and a non-waxy oil, with the waxy oil and the non-waxy oil being at least 60% of the fill material and being at a ratio of at least 10:1 (waxy oil to non-waxy oil). The semisolid fill material also includes a surfactant, one or more emulsifiers, as well as water and/or other polar solvent, with the total amount of the water and/or other polar solvent being about 10% or less of the fill material. The emulsifier can be, for example, gum Arabic, starch, or a combination, with the emulsifier being about 5% or less by weight of the semisolid softgel fill material.

In certain example aspects, the waxy oil and the non-waxy oil are present in the fill material at a ratio of at least 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1 or 30:1. In certain example aspects, the waxy oil and the non-waxy oil include at least 65% by weight of the fill material. The fill material can also include coconut oil and beeswax, with the beeswax being at least 4% by weight of the fill material. Other components of the semisolid fill material can include, for example, lecithin (such as at 4-6% by weight of the fill material) and glycerin (such as around 3-6% by weight of the fill material).

In certain example aspects, the semisolid fill material includes an active ingredient. For example, the active ingredient can be a nonpolar active ingredient, with the nonpolar active ingredient being included as part of the waxy oil or the non-waxy oil. In certain example aspects, the active ingredient of the semisolid fill material is a cannabidiol (CBD), a fish oil, a flax seed oil, a black seed oil, or combination thereof.

In certain example aspects, the surfactant of the semisolid fill material includes d-α-tocopheryl polyethylene glycol succinate (“TPGS”) or a derivative thereof. For example, the surfactant can include about 8-15% by weight of the TPGS or derivative thereof, such as about 13% by weight of the TPGS or derivative thereof.

In certain example aspects, the semisolid softgel fill material includes droplets of the waxy and non-waxy oils, the droplets having a median or average particle size of about 15 to 20 μm. In certain example aspects, the droplets have a D50 of about 15-20 μm and/or a D90 of about 45 μm.

In still further example aspects, provided is a method for manufacturing a softgel fill material, the method including providing a first mixture, the first mixture including water and/or other polar solvent and one or more emulsifiers. The method also includes providing a second mixture, the second mixture including a waxy oil, a non-waxy oil, and a surfactant. The waxy oil and/or non-waxy oil include, for example, one or more active ingredients, with the waxy oil and the non-waxy oil being present in the second mixture at a ratio of at least 7:1. To form the softgel fill material, the method includes combining the first mixture with the second mixture, wherein combining the first mixture and the second mixture includes heating the second mixture to liquify the second mixture. The liquified second mixture is then mixed with the first mixture under high shear, thereby forming the fill material. In certain example aspects, the second mixture includes a ratio of about 12:1 waxy oil to non-waxy oil, while in other aspects, the second mixture includes a ratio of about 25:1 waxy oil to non-waxy oil.

In certain example aspects, the first mixture can include at least 10% by weight of the emulsifier, with the emulsifier being, for example, gum Arabic, starch, or a combination thereof. The first mixture can also include glycerin, such as about 30-40% by weight glycerin.

In certain example aspects, the surfactant includes d-α-tocopheryl polyethylene glycol succinate (“TPGS”) or derivative thereof, with the TPGS being at least 10% of the second mixture. In certain example aspects, the second mixture further includes lecithin, such as at least 4% by weight lecithin. In certain example aspects, the non-waxy oil of the second mixture includes primarily a medium chain triglycerides (MCT) or mixture of MCTs. In certain example aspects, the second mixture can also include 3% by weight of the non-waxy component and coconut oil, such as about 50% by weight coconut oil. In still further example aspects, the second mixture includes beeswax, such as at least 5% by weight beeswax. The second mixture can also include an active ingredient, such as cannabidiol (CBD), a fish oil, a flax seed oil, a black seed oil, or combination thereof.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

FIG. 1 is an image showing conventional, oil-filled softgels that were dried according to conventional methods. As shown, the softgel shells are smooth, not too soft or mushy, and fully formed and cured.

FIG. 2A is an image showing softgels having an oil-in-water emulsion as the fill material, before the softgels are dried. As shown, the softgels were dimpled in appearance and misshapen.

FIG. 2B is an image showing softgels having the oil-in-water emulsion as in FIG. 2A, but after conventional drying. As shown, the softgels continued to have a dimpled appearance and be misshapen. The softgels were also overly soft (uncured), lacking the structural integrity of a conventional softgel.

FIG. 3A is an image showing softgels having both a waxy and non-waxy component in the fill material before drying, in accordance with certain example embodiments. As shown, the softgels were fully formed before drying and had a normal shape.

FIG. 3B is image showing softgels having both the waxy and non-waxy component in the fill material as in FIG. 3A, but after conventional drying. As shown, the softgels were fully formed after drying and retained a normal shape. The softgels also possessed structural integrity suitable for packaging and shipping.

FIG. 4 is an image showing softgels prepared as described herein in accordance with certain example embodiments, but without the waxy-oil component. Hence, the softgels did not include the ratio of waxy oil to non-waxy oil as described herein. After drying, the resulting softgels were soft (uncured) and deformed.

FIG. 5 is an image showing softgels having a higher waxy to non-waxy component ratio, in accordance with certain example embodiments. As shown, the softgels were fully formed after drying and retained a normal shape. The softgels also possessed structural integrity suitable for packaging and shipping.

FIG. 6 is an image showing softgels having a higher waxy to non-waxy component ratio, the waxy component also including beeswax, in accordance with certain example embodiments. As shown, the softgels were fully formed after drying and retained a normal shape. The softgels also possessed structural integrity suitable for packaging and shipping, with a shelf life of at least six months.

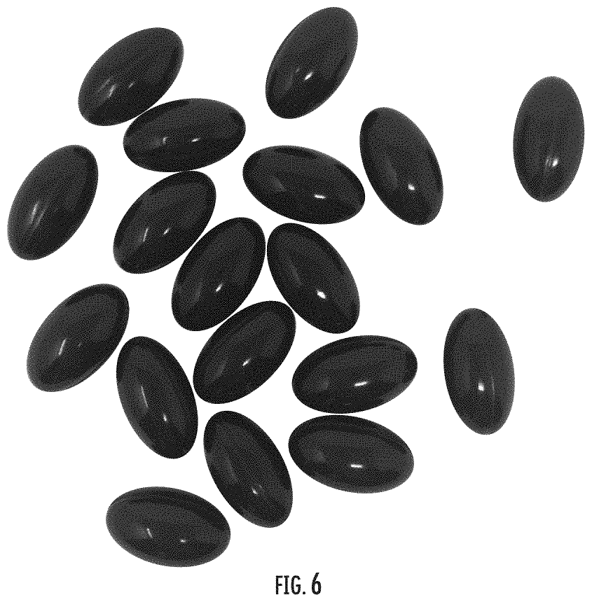

FIG. 7 is a graph showing a representative differential histogram of particle size distribution for the softgel fill material described in Example 6, in accordance with certain example embodiments. As shown, the D(50) for the sample of softgel fill material is 17.6 μm, with the majority of the particles (90% or D(90)) being 45.3 μm or less.

### DETAILED DESCRIPTION OF THE EXAMPLE EMBODIMENTS

The embodiments described herein can be understood more readily by reference to the following detailed description, examples and claims, and their previous and following description. Before the present system, devices, compositions and/or methods are disclosed and described, it is to be understood that the embodiments described herein are not limited to the specific systems, devices, and/or compositions methods disclosed unless otherwise specified, as such can, of course, vary. It is also to be understood that the terminology used herein is for describing particular aspects only and is not intended to be limiting.

Further, the following description is provided as an enabling teaching of the various embodiments in their best, currently known aspect. Those skilled in the relevant art will recognize that many changes can be made to the aspects described, while still obtaining the beneficial results of this disclosure. It will also be apparent that some of the desired benefits of the present invention can be obtained by selecting some of the features of the various embodiments without using other features. Accordingly, those who work in the art will recognize that many modifications and adaptations to the various embodiments described herein are possible and can be desirable in certain circumstances and are a part of this disclosure. Thus, the following description is provided as illustrative of the principles of the embodiments described herein and not in limitation thereof.

Reference herein to “one embodiment” or “example embodiment” means that a particular feature, structure, or characteristic described in connection with the embodiment is included in at least one embodiment of the disclosure. Appearances of the phrase “in certain example embodiments” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used herein generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way. Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

As those skilled in the art will appreciate, ranges or values can be expressed herein as from “about” one particular value, and/or to “about” another particular value. When such a range is expressed, another aspect includes from the one particular value of the range and/or to the other particular value of the range. It will be further understood that the endpoints of each of the ranges are significant both in relation to the other endpoint, and independently of the other endpoint. Similarly, when values are expressed as approximations, by use of the antecedent “about,” it will be understood that the particular value forms another aspect. In certain example embodiments, the term “about” is understood as within a range of normal tolerance in the art for a given measurement, for example, such as within 2 standard deviations of the mean. In certain example embodiments, depending on the measurement “about” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about. Further, terms used herein such as “example,” “exemplary,” or “exemplified,” are not meant to show preference, but rather to explain that the aspect discussed thereafter is merely one example of the aspect presented. Further, as used herein, the reference to a percent, such as a specified percent of a composition, refers to weight percent (or wt %).

### Overview

Disclosed herein is a semisolid softgel fill material that includes water and/or other polar solvents, along with a high amount of a nonpolar component, such as an oil. Also provided are methods and systems for producing softgels including the fill materials. The softgels produced by the described methods and systems have a normal appearance and retain their structural integrity, even when dried according to conventional methods. Further, when the fill material described herein is added to water or other liquid, as it would be following ingestion, the fill material forms small droplets, thereby enhancing digestion and absorption.

More particularly, conventional softgels including nonpolar ingredients, such as fish oil, rely on digestive enzymes to break apart that oil so that it is absorbable. For example, intestinal bile salts break large oil droplets into smaller droplets and coat the droplet to form much finer droplets. This emulsification process occurs in the intestine and is known to aid in digestion, because the finer, emulsified oil droplets have more surface area. With increased surface area, the fat-digesting enzyme pancreatic lipase acts on the surface of the oil droplet, breaking the oil down into free fatty acids and monoglycerides that are readily absorbed in the intestine. Active ingredients that are present in the oil, for example, can also be released and absorbed along with the free fatty acids and monoglycerides.

Unlike the conventional softgel that requires extensive physiological processing after ingestion, the semisolid fill material described herein can be used to deliver a fill material that is more quickly digested, thereby providing enhanced absorption. Without being bound by any particular theory, it is believed that when softgels including the fill material described herein are consumed, the water-in-oil emulsion of the fill material more quickly forms smaller oil droplets in the stomach as compared to conventional oil-containing softgel fills. The smaller oil droplets, in turn, provide more surface area upon which digestive enzymes, such as lipase, act when the emulsified fill material reaches the intestine. Hence, as compared to the consumption of a conventional oil-containing softgel, the lipase enzymes can more quickly act on the smaller particles, thereby more quickly leading to absorbable free fatty acids and monoglycerides (and to the absorption of any active ingredients associated therewith). Such rapid delivery is particularly advantageous for oils that include cannabinoids, such as CBD oil or hemp oil, where quick action may be desired. Further, even though the fill material includes water, the composition of the fill material permits softgels, including the fill material, to be dried using conventional drying techniques. That is, unlike softgels including water-based emulsions, which do not form properly, softgels including the fill material described herein have a smooth surface and with desirable structural integrity, even when dried using conventional methods.

In certain examples, to obtain a softgel fill material with one or more of the properties described herein, the fill material includes water and/or other polar solvent, along with at least two different nonpolar components, i.e., a first lipid component and a second lipid component. In certain examples, the first and second lipid components include about 65%-80% or more of total fill material. The first lipid component is a waxy oil or oil that is semisolid and/or paste-like at room temperature. The second lipid component is a non-waxy oil or an oil that is liquid at room temperature.

Further, the first lipid component and the second component are present in a ratio, for example, of about 6:1 to 12:1 in the fill material. In certain examples, the ratio is higher, such as about 8:1 to 15:1 or from about 15:1 to 28:1. With these ratios, for example, the fill material is a semisolid at room temperature or below. Additionally, the fill material also includes a surfactant, such as d-α-tocopherol polyethylene glycol succinate (“TPGS”), as well as an emulsifier. Generally, the TPGS is present in an amount of about 8%-12% or less of the fill material. In certain examples, the TPGS can be present in a higher amount, such as about 15% or more. The fill can also include other components, such as lecithin, glycerin, various additives and/or carriers. The fill material also includes an active ingredient, such as an oil or other active ingredient, which can be blended with and/or pre-mixed with either the first lipid component or the second lipid component.

To make a softgel including the semisolid fill material described herein, a water phase is combined with an oil phase to form a water-in-oil emulsion. For example, water and glycerin can be combined, with the emulsifier thereafter slowly added under high shear mixing. The emulsifier is then added. For the oil phase, the first lipid component and the second lipid component are combined, under heat, to melt the first lipid component. TPGS is added, along with lecithin. This aqueous phase and the oil phase are then mixed under shear stress to form the fill material. In certain example embodiments, the fill material is incorporated into a softgel and the softgel is dried using conventional means. Once dried, the fill material is a semisolid material as described herein, with the softgel being normal in appearance and having desirable structural properties.

### EXAMPLE EMBODIMENTS

In certain example embodiments, the fill material composition includes a first lipid (nonpolar) component and a second lipid (nonpolar) component, a surfactant, water and/or other polar component, and an emulsifier, with the first lipid component being a waxy oil present at a high amount relative to the second lipid component. Non-limiting examples of these components are described herein.

### Lipid Components

In certain examples embodiments, the first lipid component of the semisolid fill material can be any waxy oil as described herein, such as coconut oil or other waxy oil as described herein or combination of waxy oils. As a waxy oil, for example, the first lipid component generally exists as a liquid at higher temperatures but forms a paste-like semisolid at or around room temperature or below. Example waxy oils include, for example, coconut oil, cocoa butter, microcrystalline triglycerides, beeswax, palm kernel oil and other food-grade waxes and the like. In certain example embodiments, the waxy oils can include a mixture of one or more medium chain triglycerides (MCT) that have a solid/pasty appearance at room temperature or below.

In certain example embodiments, the second lipid component is a non-waxy oil that is liquid at room temperature or above. Example non-waxy oils include, for example, fish oil, black seed oil, cranberry seed oil, pumpkin seed oil, flaxseed oil, chia seed oil, CBD in MCT oil, safflower oil (CLA), or any fat based nutrient materials that are liquid at room temperature. In certain example embodiments, the second lipid component can include a mixture of one or more medium chain triglycerides that have a liquid appearance at room temperature or below.

In certain example embodiments, an active ingredient to be delivered can be solubilized within, dispersed within, and/or associated with either the first and/or second lipid component. In certain example embodiments, the active ingredient may be a nonpolar component, such as CBD oil, fish oil, or other essential or beneficial oil. For example, the nonpolar active ingredient can be omega-3 fatty acids, omega-6 fatty acids, omega-7 fatty acids, omega-9 fatty acids, conjugated fatty acids, coenzyme Q-containing active ingredients, oil soluble vitamins other than a vitamin E, carotenoid-containing active ingredients and phytochemicals. Example nonpolar active ingredients can also include, for example, nonpolar compounds containing Docosahexaenoic acid (DHA) and/or Eicosapentaenoic acid (EPA), conjugated linoleic acid (CLA), and gamma-linolenic acid (GLA), including, but not limited to, fish oil, algae oil, flaxseed oil, borage oil, oleic acid, saw palmetto extract, phytosterols, resveratrol, coenzyme Q10, vitamin D3, vitamin A palmitate, lycopene, lutein, zeaxanthin, mixtures of lutein and zeaxanthin, chia seed oil, black current oil, cranberry seed oil, blackseed oil, pumpkin oil, evening primrose oil (EPO), medium chain triglyceride oils, sunflower oil, and the like. In certain example embodiments, the active ingredient can be turmeric or astaxanthin.

The nonpolar active ingredients can also include or be associated with any nutraceutical or pharmaceutical and/or oil, such as, for example, drugs, hormones, vitamins, nutrients, which in certain examples may or may not be fat soluble. In certain example embodiments, the active is one or more cannabinoids, including for example Cannabidiol (CBD), Tetrahydrocannabinolic Acid (THCA), Tetrahydrocannabinol (THC), Cannabidiolic Acid (CBDA), Cannabinol (CBN), Cannabigerol (CBG), Cannabichromene (CBC), Tetrahydrocannabivarin (THCV), Cannabidivarin (CBDV), Cannabicyclol (CBL), Cannabivarin (CBV), Cannabichromevarin (CBCV), Cannabigerovarin (CBGV), Cannabigerol monomethyl ether (CBGM), Cannabielsoin (CBE), Cannabicitran (CBT) and the like and/or derivatives thereof.

As described herein, the first lipid component includes a higher proportion of the fill material than the second lipid component in the fill material. This ratio, for example, is believed to contribute to the semisolid nature of the fill material, such as when the fill material is included in a cooled (room temperature) and dried softgel. Hence, when the fill material is at room temperature or below, the fill material has a semisolid consistency and appearance. Yet if too much first (waxy) lipid component is included in the fill material described herein, then a softgel including the fill material that is dried using conventional processes can partially shrink and/or lack structural integrity. Conversely, when the proportion of second (non-waxy) lipid component is too high in the fill material, then the softgel can lack structural integrity and have a dimpled/shriveled appearance, a result that is believed to arise from water migration from the softgel fill to the softgel wall (with some loss to the environment).

Thus, in certain example embodiments the ratio of the first lipid component to the second lipid component in the fill material is at least 6:1, such as at least 6.5:1, 7:1, 7.5:1, 8:1, 8.5:1, 9:1, 10:1, 11:1, 12:1, 13:1, 14:1, or 15:1. In certain example embodiments, the ratio is higher, such as 16:1, 17:1, 18:1, 19:1, 20:1, 21:1, 22:1, 23:1, 24:1, 25:1, 26:1, 27:1, 28:1, 29:1 or 30:1. In certain example embodiments, the combination of the first lipid component and the second lipid component include at least about 60% of the total fill material, such as about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%. In certain example embodiments, when the active ingredient is a waxy oil at room temperature or below, the active ingredient can be combined with the first lipid component, thereby contributing to the overall oil content of the first lipid component. Additionally or alternatively, when the active ingredient is a liquid oil at room temperature or below, the active ingredient can be combined with the second lipid component, thereby contributing to the overall oil content of the second lipid component. As such, the ratios of the first lipid component and the second lipid component are maintained as described herein. In certain example embodiments, both the first and second lipid components can include an active ingredient.

### Surfactants

In addition to a first and second lipid component, in certain example embodiments the fill material includes a surfactant, such as d-α-tocopheryl polyethylene glycol succinate (“TPGS”) or a derivative thereof. As those skilled in the art will appreciate, “tocopherol” refers to the four different forms of the Vitamin E family represented by the formula below:

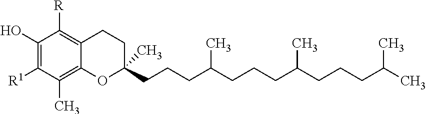

For alpha-tocopherol R and R¹═CH₃, beta-tocopherol R═CH₃ and R¹═H, gamma-tocopherol R═H and R¹═CH₃, and delta-tocopherol R and R¹═H. Each isomer can be present alone or in combination with the other isomers. Similarly, “tocotrienol” refers to the four different forms of the Vitamin E family represented by the formula below.

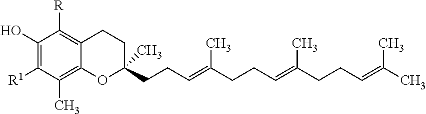

For alpha-tocotrienol R and R¹═CH₃, beta-tocotrienol R═CH₃ and R¹═H, gamma-tocotrienol R═H and R¹═CH₃, and delta-tocotrienol R and R¹═H. Each isomer can be present alone or in combination with the other isomers.

With regard to d-α-tocopheryl polyethylene glycol 1000 succinate (TPGS), TPGS has the formula:

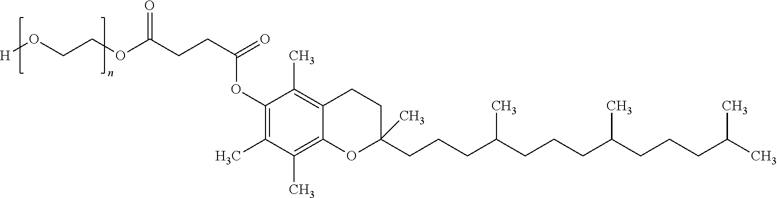

wherein the index n has an average value of 25.

Disclosed herein are also TPGS “derivatives.” The derivatives can vary by the number of ethyleneoxy units, for example, n=50, or by substituting a tocotrienol unit for the tocopherol. Other TPGS derivatives may be known by those of skill in the art.

In other example embodiments, additional and/or different surfactants that may be used in the fill material include surfactants and/or co-surfactants and/or stabilizers. Without wishing to be limited by theory, surfactants are believed to form an interfacial film between the oil and water phase of fill material thereby providing stability. Generally, the surfactants should be suitable for animal or human consumption. The following are non-limiting examples of surfactants/co-surfactants that may be used or adapted for use in preparing the disclosed compositions:

Alkyl Sulfates. The disclosed compositions can comprise one or more C₁₀-C₂₀ primary, branched chain and random alkyl sulfates having the formula ROSO₃M wherein R is a linear or branched chain comprising from 10 to 20 carbon atoms and M represents a water-soluble cation. Non-limiting examples of alkyl sulfates suitable for use in the disclosed compositions include sodium decylsulfate, sodium dodecylsulfate, sodium tetradecylsulfate, sodium hexadecylsulfate, and sodium octadecylsulfate.

Alkyl Alkoxy Sulfates. The disclosed compositions can comprise one or more C₁₀-C₁₈ alkyl alkoxy sulfates having the formula:

CH₃(CH₂)ₓ(OCH₂CH₂)_{y}OSO₃M

wherein the index x is from 9 to 17, y is from 1 to 7 and M is a water-soluble cation chosen from ammonium, lithium, sodium, potassium and mixtures thereof. A non-limiting example includes sodium dodecyl diethoxy sulfate having the formula:

CH₃(CH₂)₁₁(OCH₂CH₂)₂OSO₃Na.

Alkyl alkoxy sulfates are also commercially available as a mixture of ethoxylates, for example, sodium laureth sulfate is available as a mixture of ethoxylates, i.e., the index y is from 2 to 4. Other suitable examples include sodium laureth-2 sulfate having an average of 2 ethoxylates and a C₁₂ linear alkyl chain. Sodium laureth-2 is available as Texapon™ N 56 from Cognis Corp. Further examples of alkyl alkoxy sulfates includes sodium laureth-1 sulfate, sodium laureth-3 sulfate, sodium laureth-4 sulfate, sodium myreth-2 sulfate and sodium myreth-3 sulfate.

Alkenyl Sulfonates. The disclosed compositions can comprise one or more C₁₀-C₁₈ alkenyl sulfonates (α-olefin sulfonates) having the formula:

CH₃(CH₂)_{z}CH═CHSO₃M

wherein the index z is from 7 to 15 and M is a water-soluble cation chosen from ammonium, lithium, sodium, potassium and mixtures thereof. Olefin sulfonates are commercially available as a mixture of alkenyl chains, for example, sodium C₁₄-C₁₆ olefin sulfonate Bio-Terge™ AS-40 available from Stepan. Further non-limiting examples of alkenyl sulfonates include C₁₂-C₁₆ olefin sulfonates and C₁₄-C₁₈ olefin sulfonates. Another example is C₁₂-C₁₅ pareth-15 sulfonate available as Avanel™ S 150 CG.

Alkyl Alkoxy Carboxylates. The disclosed compositions can comprise one or more C₁₀-C₁₈ alkyl alkoxy carboxylates having the formula:

CH₃(CH₂)ₓ(OCH₂CH₂)_{y}CO₂M

wherein the index x is from 9 to 17, y is from 1 to 5 and M is a water-soluble cation chosen from ammonium, lithium, sodium, potassium and mixtures thereof. A non-limiting example includes sodium dodecyl diethoxy carboxylate having the formula:

CH₃(CH₂)₁₁(OCH₂CH₂)₂CO₂Na.

Alkyl alkoxy carboxylates are also commercially available as a mixture of ethoxylates, for example, sodium laureth sulfate is available as a mixture of ethoxylates, i.e., the index y is from 2 to 4. Other suitable examples include sodium laureth-2 sulfate having an average of 2 ethoxylates and a C₁₂ linear alkyl chain. Sodium laureth-2 is available as Texapon™ N 56 from Cognis Corp. Further examples of alkyl alkoxy sulfates include sodium laureth-1 sulfate, sodium laureth-3 sulfate, sodium laureth-4 sulfate, sodium myreth-2 sulfate and sodium myreth-3 sulfate.

Isethionate Esters of Alkyl Alkoxy Carboxylic Acids. The disclosed compositions can comprise one or more C₁₀-C₁₈ isethionate esters of alkyl alkoxy carboxylates having the formula:

CH₃(CH₂)ₓ(OCH₂CH₂)_{y}OCH₂C(O)OCH₂CH₂SO₃M

wherein the index x is from 9 to 17, the index y is from 1 to 5 and M is a water-soluble cation. Isethionate esters of alkyl alkoxy carboxylates are described in U.S. Pat. No. 5,466,396 the disclosure of which is included herein by reference in its entirety.

Alkyl Carboxyamides. The disclosed compositions can comprise one or more C₁₀-C₁₈ alkyl carboxyamides having the formula:

CH₃(CH₂)ₓC(O)NR(CH₂)_{y}CO₂M

wherein R is hydrogen or methyl the index x is from 9 to 17, the index y is from 1 to 5 and M is a water-soluble cation. A non-limiting example of an alkyl carboxyamide suitable for use in the disclosed compositions includes potassium cocoyl glycinate available as AMILITE™ GCK-12 from Ajinomoto. A further example includes compounds wherein R is methyl, for example, sodium cocoyl sarcosinate.

Alkyl Amide Betaines. One category of zwitterionic surfactants relates to C₁₀-C₁₆ alkyl amide betaines having the formula:

CH₃(CH₂)_{w}C(O)NH(CH₂)ᵤN⁺(CH₃)₂(CH₂)ₜCO₂⁻

wherein the index w is from 9 to 15, the index u is from 1 to 5 and the index t is from 1 to 5. Non-limiting examples of betaine surfactants includes {[3-(decanoylamino)ethyl]-(dimethyl)-ammonio}acetate, {[3-(decanoylamino)ethyl](dimethyl)ammonio}-acetate, {[3-(dodecanoyl-amino)ethyl](dimethyl)ammonio}acetate, {[3-(dodecanoylamino)propyl]-(dimethyl)-ammonio}acetate, {[3-(dodecanoylamino)-butyl](dimethyl)ammonio}acetate, {[3-(tetra-decanoylamino)ethyl](dimethyl)-ammonio}acetate, {[3-(tertadecanoylamino)-propyl](dimethyl)ammonio}acetate, {[3-(hexadecanoylamino)ethyl](dimethyl)-ammonio}acetate, and {[3-(hexa-decanoylamino)propyl](dimethyl)ammonio}acetate.

Alkyl Amide Sultaines. Another category of zwitterionic surfactants relates to C₁₀-C₁₆ alkyl amide sultaines having the formula:

CH₃(CH₂)_{w}C(O)NH(CH₂)ᵤN⁺(CH₃)₂(CH₂)ₜSO₃⁻

wherein the index w is from 9 to 15, the index u is from 1 to 5 and the index t is from 1 to 5. Non-limiting examples of sultaine surfactants includes {[3-(decanoylamino)ethyl]-(dimethyl)-ammonio}methanesulfonate, {[3-(decanoylamino)ethyl](dimethyl)ammonio}-methanesulfonate, {[3-(dodecanoyl-amino)ethyl](dimethyl)ammonio}methanesulfonate, {[3-(dodecanoylamino)-propyl](dimethyl)ammonio}methanesulfonate, {[3-(dodecanoyl-amino)butyl](dimethyl)-ammonio}methanesulfonate, {[3-(tetradecanoylamino)ethyl]-(dimethyl)ammonio}methane-sulfonate, {[3-(tertadecanoylamino)propyl](dimethyl)-ammonio}methanesulfonate, {[3-(hexadecanoylamino)ethyl](dimethyl)ammonio}-methanesulfonate, and{[3-(hexadecanoylamino)propyl](dimethyl)ammonio}-methanesulfonate.

Alkyl Hydroxy Sultaines. A further category of zwitterionic surfactants relates to C₁₀-C₁₆ alkyl hydroxy sultaines having the formula:

CH₃(CH₂)_{w}N⁺(CH₃)₂CH₂CHOHCH₂SO₃⁻

wherein the index w is from 9 to 15. Non-limiting examples of alkyl hydroxy sultaine surfactants includes 3-[dodecyl(dimethyl)azaniumyl]-2-hydroxypropane-1-sulfonate (lauryl hydroxysultaine), 3-[tetradecyl(dimethyl)azaniumyl]-2-hydroxypropane-1-sulfonate (myristyl hydroxysultaine), (Z)-{dimethyl[3-(octadec9-enamido)propyl]ammonio}-methanesulfonate (oleyl hydroxysultaine), and the like.

In certain example embodiments, the total amount of surfactant in the fill material is about 8%, 9%, 10%, 11%, 12%, 13%, 14%, or 15% of the fill material. For example, when TPGS and/or a derivative thereof is the only surfactant, the amount of TPGS in the fill material comprises about 8%, 9%, 10%, 11%, 12%, 13%, 14%, 15%, 16%, 17%, 18%, 19%, or 20% of the fill material. In certain example embodiments, the TPGS and/or derivative thereof comprises less than about 10% of the fill material, such as about 4%, 5%, 6%, 7%, 8%, or 9% of the fill material. In certain example embodiments, the amount of surfactant, such as TPGS or derivative thereof, may be as low as less than about 2% of the fill material, such as about 0.1%, 0.2%, 0.3%, 0.4% 0.5%, 0.6% 0.7%, 0.8%, 0.9%, 1.0%, 1.1%, 1.2%, 1.3%, 1.4%, 1.5%, 1.6%, 1.7%, 1.8%, or 1.9%.

### Emulsifiers

In certain example embodiments, the semisolid fill material includes an emulsifier. As those skilled in the art will appreciate, emulsifiers refer to an ingredient or combination of ingredients that prevents the separation of emulsions. Generally, emulsifiers have a polar group with an affinity for water (hydrophilic) and a nonpolar group that is attracted to oil (lipophilic). They are absorbed at the interfaces of the two substances, providing an interfacial film acting to stabilize the emulsion. The hydrophilic/lipophilic properties of emulsifiers are affected by the structure of the molecule. These properties are identified by the hydrophilic/lipophilic balance (HLB) value. Low HLB values indicate greater lipophilic tendencies which are used to stabilize water-in-oil emulsions. High HLB values are assigned to hydrophilic emulsifiers, typically used in oil-in-water emulsions. These values are derived from simple systems. Because different types of water-in-oil products can be prepared according to the method described herein often contain other ingredients that affect the emulsification properties, the HLB values may not always be a reliable guide for emulsifier selection.

In certain example embodiments, the emulsifiers are food-grade emulsifiers suitable for animal or human consumption. Example emulsifiers include, mono- and diglycerides, glycerides, monoglyceride derivatives, and fatty acid derivatives, DATEM (diacetyltartaric acid ester of monoglycerides), sorbitan esters, sugar esters and the like. In certain examples embodiments, a mixture of one or more emulsifiers can be used to achieve the fill material.

One category of emulsifiers includes, for example, C₁₄-C₂₂ fatty alcohols, non-limiting examples of which are chosen from 1-tetradecanol (myristyl alcohol), 1-hexadecanol (cetyl alcohol), cis-9-hexadecen-1-ol (plamitoleyl alcohol), 1-octadecanol (stearyl alcohol), cis-9-octadecen-1-ol (oleyl alcohol), trans-9-octadecen-1-ol (elaidyl alcohol), 1-eicosanol (arachidyl alcohol), and 1-docosanol (behenyl alcohol). Further non-limiting examples of emulsifiers include esters of C₁₄-C₂₂ fatty alcohols and inorganic acids chosen from di-1-tetradecanyl phosphate (di-myristyl phosphate), di-1-hexadecanyl phosphate (di-cetyl phosphate), di-cis-9-hexadecen-1-yl phosphate (di-plamitoleyl phosphate), di-1-octadecanyl phosphate (di-stearyl phosphate), di-cis-9-octadecen-1-yl phosphate (di-oleyl phosphate), di-trans-9-octadecen-1-yl phosphate (di-elaidyl phosphate), di-1-eicosanyl phosphate (di-arachidyl phosphate), di-1-docosanyl phosphate (di-behenyl phosphate), 1-tetradecanyl sulfate (myristyl sulfate), 1-hexadecanyl sulfate (cetyl sulfate), cis-9-hexadecen-1-yl sulfate (plamitoleyl sulfate), 1-octadecanyl sulfate (stearyl sulfate), cis-9-octadecen-1-yl sulfate (oleyl sulfate), trans-9-octadecen-1-yl sulfate (elaidyl sulfate), 1-eicosanyl sulfate (arachidyl sulfate), and 1-docosanyl sulfate (behenyl sulfate).

Another category of emulsifiers includes, for example, glyceryl monostearate, glyceryl monopalmitate, glyceryl monooleate (monostearin, monopalmitin, monoolein), lactic acid esters of mono- and diglycerides of fatty acids, citric acid esters of mono- and diglycerides of fatty acids, mono- and diacetyl tartaric acid esters of mono- and diglycerides of fatty acids, sucrose esters of fatty acids, i.e., mono-, di- and triesters of sucrose with fatty acids.

A further category of emulsifiers includes fatty acid esters of propane-1,2-diol. Non-limiting examples include 1-hydroxypropan-2-yl dodecanoate, 2-hydroxypropyl dodecanoate, propane-1,2-diyl didodecancoate, 1-hydroxypropan-2-yl tetradecanoate, 2-hydroxypropyl tetradecanoate, propane-1,2-diyl ditetradecancoate, 1-hydroxypropan-2-yl hexadecanoate, 2-hydroxypropyl hexadecanoate, and propane-1,2-diyl dihexadecancoate.

A further category of emulsifiers includes C₈-C₁₈ alkylglycosides having the formula:

CH₃(CH₂)_{q}O[G]ₚH

wherein G represents a monosaccharide residue chosen from glucose, fructose, mannose, galactose, talose, allose, altrose, idose, arabinose, xylose, lyxose, ribose and mixtures thereof, the index p is from 1 to 4, the index q is from 7 to 17. The following are non-limiting examples of alkyl glucoside surfactants include (2R,3S,4S,5R,6R)-2-(hydroxymethyl)-6-octooxyoxane-3,4,5-triol (octyl glucoside, n-octyl-b-D-glucoside), (2R,3R,4S,5S,6R)-2-decoxy-6-(hydroxymethyl)tetra-hydropyran-3,4,5-triol (decyl glucoside, n-decyl-b-D-glucoside), and (2R,3R,4S,5S,6R)-2-dodecoxy-6-(hydroxymethyl)tetrahydropyran-3,4,5-triol (dodecyl glucoside, lauryl glucoside, n-dodecyl-b-D-glucoside). One example of a suitable admixture of C₈-C₁₆ alkylglycosidyl nonionic surfactants is PLANTACARE™ 818 UP available from Cogins Chemical Co.

Still another category of emulsifiers includes polyoxyethylene glycol alkyl ethers having the formula:

RO(CH₂CH₂O)ₙH

wherein R is a linear or branched alkyl group having from 6 to 20 carbon atoms and n is an integer of about 2 to about 20.

One example of suitable ethoxylate alcohols is the NEODOL™ ethoxylated alcohols from Shell Chemicals. NEODOL™ 23-1 comprises a mixture of R units that are C₁₂ and C₁₃ in length with an average of 1 ethoxy unit. Non-limiting examples of ethoxylated alcohols include NEODOL™ 23-1, NEODOL™ 23-2, NEODOL™ 23-6.5, NEODOL™ 25-3, NEODOL™ 25-5, NEODOL™ 25-7, NEODOL™ 25-9, PLURONIC™ 12R3, and PLURONIC™ 25R2 available from BASF.

In certain example embodiments, the emulsifier includes polyoxyethylene glycol alkyl ethers having the formula:

RO(CH₂CH(CH₃)O)ₙH

wherein R is a linear or branched alkyl group having from 6 to 20 carbon atoms and n is an integer of about 2 to about 20.

In certain example embodiments, the emulsifier includes polyoxyethylene polyoxypropylene block copolymers known as “poloxamers” having the formula:

HO(CH₂CH₂)_{y1}(CH₂CH₂CH₂O)_{y2}(CH₂CH₂O)_{y3}OH.

These are nonionic block copolymers composed of a polypropyleneoxy unit flanked by two polyethyleneoxy units. The indices y¹, y², and y³ have values such that the poloxamer has an average molecular weight of from about 1000 g/mol to about 20,000 g/mol. These are also well known by the trade name PLURONICS™. These compounds are commonly named with the word Poloxamer followed by a number to indicate the specific co-polymer, for example Poloxamer 407 having two PEG blocks of about 101 units (y¹ and y³ each equal to 101) and a polypropylene block of about 56 units. This category of emulsifiers is commercially available, for example, under the trade name LUTROL™ F-17 available from BASF.

A further example of emulsifiers includes alkyl amides that are ethoxylate, propoxylated, or mixtures thereof, having the formula:

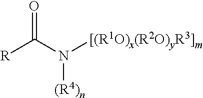

wherein R is C₇-C₂₁ linear alkyl, C₇-C₂₁ branched alkyl, C₇-C₂₁ linear alkenyl, C₇-C₂₁ branched alkenyl, and mixtures thereof. R¹ is ethylene; R² is C₃-C₄ linear alkylene, C₃-C₄ branched alkylene, and mixtures thereof; in some iterations R² is 1,2-propylene. Emulsifiers that comprise a mixture of R¹ and R² units can comprise from about 4 to about 12 ethylene units in combination with from about 1 to about 4 1,2-propylene units. The units can be alternating or grouped together in any combination suitable to the formulator. In one iteration, the ratio of R¹ units to R² units is from about 4:1 to about 8:1. In another iteration, a R² unit (i.e., 1,2-propylene) is attached to the nitrogen atom followed by the balance of the chain comprising from 4 to 8 ethylene units. R³ is hydrogen, C₁-C₄ linear alkyl, C₃-C₄ branched alkyl, and mixtures thereof; preferably hydrogen or methyl, more preferably hydrogen. R⁴ is hydrogen, C₁-C₄ linear alkyl, C₃-C₄ branched alkyl, and mixtures thereof. When the index m is equal to 2 the index n must be equal to 0 and the R⁴ unit is absent and is instead replaced by a —[(R¹O)ₓ(R²O)_{y}R³] unit. Further, the index m is 1 or 2, the index n is 0 or 1, provided that when m is equal to 1, n is equal to 1; and when m is 2 n is 0; in one example, m is equal to 1 and n is equal to one, resulting in one —[(R¹O)ₓ(R²O)_{y}R³] unit and R⁴ being present on the nitrogen. The index x is from 0 to about 50, in one embodiment from about 3 to about 25, in another embodiment x is from about 3 to about 10. The index y is from 0 to about 10, in one example y is 0; however, when the index y is not equal to 0, y is from 1 to about 4. In one embodiment all of the alkyleneoxy units are ethyleneoxy units.

In certain example embodiments, the total amount of emulsifier in the fill material is less than about 10% of the fill material, such as about 1%, 2%, 3%, 4%, 5%, 6%, 7%, 8%, or 9%. In certain example embodiments, two or more emulsifiers may be used in the fill material. For example, when the emulsifier is gum Arabic or starch, the amount of these components can be less than about 2%, such as about 0.1%, 0.2%, 0.3%, 0.4% 0.5%, 0.6% 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, or 2.2% of the fill material. In certain example embodiments, the emulsifier is a starch-based emulsifier. One example emulsifier is EmulTru™ (from Cargil™), which is characterized as an instantized lipophilic starch. For example, the final fill material may include about 1-5% of a starch-based emulsifier, such as about 1.0%, 1.5%, 2.0%, 2.5%, 3.0%, 3.5%, or 4.0%, 4.5%, or 5% of the emulsifier (e.g., EmulTru™).

### Additives

In certain example embodiments, the fill material compositions provided herein can include one or more additives. For example, the fill material may include one or more adjunct components or additives are also included, such as vitamins. The vitamins can be added directly, for example, tocopherol, or the vitamin can be added in a releasable form, for example, ascorbyl palmitate. Other suitable adjunct components include thickening agents, for example, starches, vegetable gums, pectin or proteins. Non-limiting examples of thickening agents include alginic acid, sodium alginate, potassium alginate, ammonium alginate, calcium alginate, agar, carrageenan, locust bean gum pectin, gelatin, and xanthan gum. Other adjunct components or additives include essential oils, fragrances, flavorants, colorants, and glycerin. Generally, such additives are suitable for human or animal consumption.

In certain example embodiments, the fill material can include lecithin and/or other related amphiphilic glycerophospholipids. For example, components such as lecithin can be added to the oil phase of the fill material during preparation of the fill material. As such, the resultant fill material can include about 3%, 4%, 5%, 6%, or 7% of the lecithin and/or related amphiphilic glycerophospholipid. Without wishing to be bound by any particular theory, it is believed that inclusion of such amphiphilic glycerophospholipids (e.g., lecithin) lubricates the oil phase during manufacture of the fill material, thereby further facilitating manufacture of softgels including the fill material. In certain example embodiments, the lubricant can be polyethylene glycol or derivative thereof and/or a detergent such as polysorbate based detergent (e.g., Tween-80).

In certain example embodiments, the fill material can include glycerin and/or other related polyol component that has similar properties to glycerin. For example, the compositions may include about 1-15% or less of glycerin, such as about 1%, 2%, 3%, 4%, 5%, 6%, 7%, 8%, 9%, 10%, 11%, 12%, 13%, 14%, or 15% glycerin. In certain example embodiments, the fill material can include glycerin and/or other related polyol component at about 5-6% of the fill material. As those skilled in the art will appreciate, glycerin acts as a humectant, solvent, and sweetener, and may help preserve foods.

### Example Compositions & Properties

In addition to the above components, the semisolid fill material includes water and/or other polar solvent. For example, the fill material can include the components described above, as well as other additives, with the balance of the fill material being water and/or other polar solvent.

In certain example embodiments, the fill material includes about 70-95% total nonpolar component, such as about 60%, 65%, 70%, 75%, 80%, 85%, 90%, or about 95% of the nonpolar (waxy, first lipid component) and nonpolar (non-waxy, second lipid) components. In certain example embodiments, the combination of first and second lipid components are at ratios of about 6:1 to 12:1 first (waxy) lipid component to the second (liquid) lipid component. In certain example embodiments, the ratio of waxy to non-waxy components can be higher, such as about 12:1, 13:1, 14:1, or 15:1. In such example embodiments, the fill material can also include about 3-20% of a surfactant, such as about 3-20% TPGS or about 10% TPGS. Further included, for example, can be one or more emulsifiers, such as Gum Arabic and/or starch at about 1-4% or less of the fill material. In certain example embodiments, a lubricant such as lecithin can be included, such as with the lecithin being about 4-6% of the fill material. Such example fill materials can also include about 1-10% or less of glycerin, such as about 5-6%, with the balance (about 4-10%) being water or other polar solvent. For example, the fill material may include less than about 10% water and/or other polar solvent, such as about 1%, 2%, 3%, 4%, 5%, 6%, 7%, 8%, or 9% water and/or other polar solvent.

In certain example embodiments, the fill material includes about 88% total nonpolar component, i.e., the combination of first and second lipid components at ratios of about 8:1 first (waxy) lipid component to the second (liquid) lipid component. In such example embodiments, the fill material also includes about 9% TPGS. Further included, for example, is an emulsifier, such as Gum Arabic and/or starch at about 1% of the fill material, along with lecithin at about 4% of the fill material. Such example fill materials also include about 4% or less of glycerin, with the balance (about 5%) being water and/or other polar solvent.

In certain example embodiments, the fill material includes about 70% total nonpolar component, i.e., the combination of first and second lipid components at ratios of about 12:1 first (waxy) lipid component to the second (liquid) lipid component. In such example embodiments, the fill material also includes about 14% TPGS. Further included, for example, is an emulsifier, such as a starch-based emulsifier, such as EmulTru™, at about 2-3% of the fill material, along with lecithin at about 5% of the fill material. Such example fill materials also include about 5% or less of glycerin, with the balance (about 5% or less) being water and/or other polar solvent.

In certain example embodiments, the fill material includes about 70-95% total nonpolar component, such as about 60%, 65%, 70%, 75%, 80%, 85%, 90%, or about 95% of the nonpolar (waxy, first lipid component) and nonpolar (non-waxy, second lipid) components. In certain example embodiments, the combination of first and second lipid components are at ratios of about 20:1 to 30:1 first (waxy) lipid component to the second (liquid) lipid component, such as about 25:1. In such example embodiments, the fill material can also include about 10-20% of a surfactant, such as about 10-20% TPGS or about 13% TPGS. Further included, for example, can be one or more emulsifiers, such as Gum Arabic and/or starch at about 1-4% or less of the fill material. In certain example embodiments, a lubricant such as lecithin can also be included, such as with the lecithin being about 4-6% of the fill material. Such example fill materials can also include about 1-10% or less of glycerin, such as about 5-6%, with the balance (about 4-10%) being water or other polar solvent. For example, the fill material may include less than about 10% water and/or other polar solvent, such as about 1%, 2%, 3%, 4%, 5%, 6%, 7%, 8%, or 9% water and/or other polar solvent. In certain example embodiments, beeswax is also included in the fill material, the beeswax being about 4%, 5%, 6%, 7%, 8%, 9%, or 10% of the fill material. In certain example embodiments, the beeswax can be substituted with another tough wax, such as soy wax, candelilla wax, paraffin wax, carnauba wax, olive wax, rice bran wax, sunflower wax or the like, or combinations thereof.

In certain example embodiments, the second (non-waxy) lipid component of the fill material can be a medium chain triglyceride (MCT). For example, the MCT can be present from about 3% to about 7%, such as about 3%, 4%, 5%, 6%, or 7% of the fill material. In other example embodiments, the amount of MCT can be lower, such as a about 2% to 5%, such as about 2%, 3%, 4%, or 5% of the fill material. In certain example embodiments, the second (non-waxy) lipid can include MCT as well as one or more other non-waxy lipid components, the combination of the MCT and the one or more other non-waxy lipid components comprising the second (non-waxy) lipid component of the fill material as described herein. In certain example embodiments, the non-waxy MCT may be caproic acid (or hexanoic) acid and/or caprylic acid (or octanoic) acid.

While the fill material described herein is ideally suited for incorporation into a softgel, such as a softgel that is to be dried according to conventional methods and/or a softgel where improved digestion properties are desired, the fill material may also be used with and/or integrated into a variety of consumable products and foodstuffs. For example, the compositions provided herein can be used in nutraceutical products, such as fish oil or flaxseed oil health supplements, as well as supplements containing CBD or other cannabinoids as described herein. The fish oil, flaxseed oil, or CBD oil, for example, can be included as the liquid lipid component in the emulsion as described herein, and the prepared fill material can be used as a supplement by the end user. The prepared fill material can also include additives, for example, to enhance shelf-life, flavor, and product appearance. In certain example embodiments, the fill material compositions may be included within a pharmaceutical/nutraceutical delivery form, such as within a softgel as described herein or with a capsule-based delivery system. When prepared according to the methods and systems described herein, the fill material has a semisolid or paste-like appearance. As such, the fill material is not a liquid at room temperature.

In certain example embodiments, the nonpolar component of the softgel fill material exists as droplets that are interspersed among the low amount of water of the softgel fill material. The droplets, for example, can exist as different sizes that can be determined by a variety of conventional methods, which can be chosen by the formulator. In certain example embodiments, the particle size may be a “mean” particle size of the droplets that are dispersed in the nonpolar (oil) phase of the disclosed dispersions. For example, the mean particle size may be a volume weighted mean. In certain example embodiments, Light Amplification by Stimulated Emission of Radiation using MalvemSize LASER Diffractor (e.g., Model S and Model 2000 instruments) can be used to determine particles size. As used herein, the term “median particle size” refers to the average diameter of the droplets that are dispersed in the nonpolar (oil) phase of the disclosed dispersions. Further, and as those skilled in the art will appreciate, particle size is typically described relative to a particle size distribution, such as a 10%, 50%, 90% size value, which indicates the specific size median that 10%, 50%, or 90% of the particles within the distribution is smaller than. For example, Particle Size Distribution D50 is also known as the median diameter or the medium value of the particle size distribution, as this is the value of the particle diameter at 50% in the cumulative distribution. For example, if D50=17 μm, then 50% of the particles in the sample are larger than 17 um, and 50% smaller than 17 μm. As such, D50 is usually used to represent the particle size of group of particles. Likewise, if D90=45 μm, then 90% of the particles in the sample are larger than 45 um and 10% smaller than 45 μm.

In certain example embodiments, nonpolar droplets of the softgel fill material may have a mean or median particle diameter (or size) of between about 5 μm to about 50 μm. That is, the mean or median particle diameter of the nonpolar droplets of the softgel fill material may be about 5 μm, 10 μm, 15 μm, 20 μm, 25 μm, 30 μm, 35 μm, 40 μm, 45 μm, or 50 μm values therebetween, such as about 11 μm, 12 μm, 13 μm, 14 μm, 15 μm, 16 μm, 17 μm, 18 μm, 19 μm, 20 μm, 21 μm, 22 μm, 23 μm, 24 μm, or 25 μm. For example, the D(50) can be about 5 μm, 10 μm, 15 μm, 20 μm, 25 μm, 30 μm, 35 μm, 40 μm, 45 μm, or 50 μm values therebetween, such as about 11 μm, 12 μm, 13 μm, 14 μm, 15 μm, 16 μm, 17 μm, 18 μm, 19 μm, 20 μm, 21 μm, 22 μm, 23 μm, 24 μm, or 25 μm. In certain example embodiments, the majority of particles in the fill material can be between about 30 μm to about 60 μm, such as between about 35 μm to about 55 μm or about 40 μm, 41 μm, 42 μm, 43 μm, 44 μm, 45 μm, 46 μm, 47 μm, 48 μm, 49 μm, 50 μm. For example, the D(90) can be about 30 μm to about 60 μm, such as between about 35 μm to about 55 μm or about 40 μm, 41 μm, 42 μm, 43 μm, 44 μm, 45 μm, 46 μm, 47 μm, 48 μm, 49 μm, 50 μm.

In certain example embodiments, without being bound by any particular theory, it is believed that when the fill material provided herein is added to water or other liquid, such as would be the case when a softgel including the fill material is ingested with water, the fill material forms small oil droplets within the water. Such droplets, for example, may have an average diameter of less than or equal to 5 μm. In certain example embodiments the average diameter is less than or equal to 4.5 μm. In certain example embodiments, the average diameter is less than or equal to 4 μm. In a further embodiment the average diameter is less than or equal to 3.5 μm. In further embodiments the average diameter is less than or equal to 3 μm. In a yet another embodiment the average diameter is less than or equal to 2.5 μm. In a still yet further embodiment the average diameter is less than or equal to 2 μm. In a still yet another embodiment the average diameter is less than or equal to 1.5 μm.

In a yet still further embodiment, when the softgel fill material is added to water or other liquid, the average diameter may be less than or equal to 1 μm. For example, the average diameter may have any value from 5 nanometers (nm) to 5 μm. The average diameter can have any value in the recited range. For example, in the range 5 nm to 100 nm, the average can be any value, i.e., 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 55 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm and 100 nm.

In certain example embodiments, when the softgel fill material is added to water or other liquid, the droplets described herein may have a particle size distribution, D50, from about 50 nm to about 5 μm. In one embodiment the particle size distribution, D50, from about 50 nm to about 5 μm. In another embodiment the particle size distribution, D50, from about 100 nm to about 5 μm. In a further embodiment the particle size distribution, D50, from about 100 nm to about 1 μm. In a still further embodiment the particle size distribution, D50, from about 1 μm to about 5 μm. In a yet another embodiment the particle size distribution, D50, from about 250 nm to about 1 μm. In a still yet further embodiment the particle size distribution, D50, from about 1 μm to about 5 μm. In a still yet another embodiment the particle size distribution, D50, from about 500 nm to about 5 μm. In a yet still further embodiment the particle size distribution, D50, from about 500 nm to about 1 μm. In yet still further embodiments, the particle size distribution, D50, is about 0.3 μm, 0.4 μm, 0.5 μm, 0.6 μm, 0.7 μm, 0.8 μm, 0.9 μm, 1.0 μm, 1.5 μm, 2.0 μm, 2.5 μm, 3.0 μm, 3.5 μm, 4.0 μm, 4.5 μm, or 5.0 μm.

In certain example embodiments, the particle size distribution, D50, of the droplets when the softgel fill material is added to water or other liquid can have any value from 5 nanometers (nm) to 5 μm in the recited range. For example, in the range 50 nm to 1 μm, the average can be any value, i.e., 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 550 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm and 1 μm.

### Example Methods & Systems

In certain example embodiments, provided are methods and systems for manufacturing a softgel fill material described herein. Generally, the methods and systems include combining a first and second mixture under shear stress to produce the semisolid fill material described herein. Also provided are methods and systems for manufacturing a softgel including the fill material.

For example, to make the fill material, a first mixture is provided, the first mixture being an aqueous phase. The first mixture includes, for example, water and/or other polar solvent, along with one or more emulsifiers as described herein, such as gum Arabic and/or starch. In certain example embodiments, the first mixture also includes glycerin and/or related polyol component. More particularly, in certain example embodiments the first mixture (or aqueous phase) includes about 40-100% water and/or other polar solvent, such as about 40%, 45%, 50%, 55%, 65%, 70%, 75%, 80%, 85%, 90%, 95% water. In other example embodiments, the amount of water is reduced, such as to about 30%, 35%, or 40% of the first mixture. In certain example embodiments, glycerin and/or related polyol components can be added to the water. For example, the first mixture can contain about 40%-60% water and about 40%-50% glycerin. In other example embodiments, the first mixture can contain about 30%-50% water and about 30%-40% glycerin, such as about 35% water and 35% glycerin.

The first mixture can also include about 10% of one or more emulsifiers. In other example embodiments, the first mixture can include higher amounts of emulsifier, such as about 15-25% by weight of the emulsifier, for example, about 20% emulsifier or 30%. In certain example embodiments, the first mixture includes about 50% water, about 50% glycerin, about 4% gum Arabic, and about 6% starch-based emulsifier. In certain example embodiments, the first mixture includes about 40% water, about 40% glycerin, and about 20% starch-based emulsifier. In other example embodiments, the first mixture includes about 35% water, about 35% glycerin or related polyol, and about 30% of an emulsifier. In such example embodiments, the water and the glycerin can be mixed together at room temperature, with the emulsifier(s) being slowly added thereafter under high shear. The first mixture is mixed until uniform before being combined with the second mixture as described herein.

In addition to the first mixture, also provided is a second mixture, the second mixture being an oil phase. The second mixture includes, for example, a waxy oil, a non-waxy oil, and a surfactant as described herein. The waxy oil and/or non-waxy oil can also include one or more active ingredients as described herein, such as a fish oil and/or CBD oil. And to achieve a softgel fill material with the beneficial properties described herein, in certain example embodiments, the waxy oil to non-waxy oil component are in a ratio of about 6:1 to 9:1, such as about 8:1 in the second mixture, with this same ratio being present in the final fill material described herein (as the aqueous phased does not add lipid components to the fill material). In certain other example embodiments, the ratio of waxy oil to non-waxy oil is higher in the second mixture, such as about 8:1 to 15:1, with this same ratio being present in the fill material described herein. In still other example embodiments, the ratio of waxy oil to non-waxy oil is even higher in the second mixture, such as about 15:1 to 30:1, such as about 25:1, with this same ratio being present in the final fill material as described herein. In certain example embodiments, the second mixture can also include a surfactant, such as TPGS, along with a lubricant such as lecithin. For example, the second mixture can include about 7-15% TPGS, such as about 10% TPGS. In other example embodiments, the second mixture can include a higher amount of TPGS, such as about 10-20% TPGS, for example, about 15% TPGS. In certain example embodiments, the second mixture can include about 4-8% lecithin, such as about 5 to 6% lecithin.

In certain example embodiments, before combining the components of the second mixture, an active ingredient can be included with either the waxy and/or non-waxy component. For example, if the active ingredient is a waxy oil, the active waxy oil can be mixed with the waxy oil, such as by warming (melting) the waxy oil and the active waxy oil and mixing the two components together. Additionally, or alternatively, the active waxy oil can be combined with the non-waxy oil, such as by heating (melting) the active waxy oil and combining it with the non-waxy component. Likewise, in certain example embodiments, if the active ingredient is a liquid oil at room temperature, the active liquid oil can be mixed with the waxy oil, such as by warming (melting) the waxy oil and the active liquid oil and mixing the two components together. Additionally or alternatively, the active liquid oil can be combined with the non-waxy oil, mixing the two components together. Regardless, the ratio of the first lipid component (waxy oil) to the second lipid (liquid oil) component are retained as described herein. In certain example embodiments, the liquid oil may be the active ingredient. For example, the non-waxy oil may be fish oil. Additionally or alternatively, the waxy oil may be the active ingredient. It will also be appreciated, in view of this disclosure, that the waxy oil and/or the non-waxy oil can be a combination of different waxy oils and non-waxy oils, respectively. For example, the waxy oil may be a combination of coconut oil and beeswax, with the beeswax being 3%, 4%, 5%, 6%, 7%, 8%, 9% or more of the waxy component, with the reminder of the waxy oil component being coconut oil.

To prepare the second mixture, once any active ingredient is included in the waxy and/or non-waxy components, the waxy oil and the non-waxy oil are combined with the surfactant and optionally the lubricant as described herein. For example, the waxy oil and the non-waxy oil are combined with TPGS and lecithin as described herein, and the mixture is heated until the mixture is liquid. In certain example embodiments, the mixture is heated to above 30° C. to melt the waxy component, such as about 31, 32, 33, 34, 35, 36, 37, 38, 39, or 40° C., thereby forming the second mixture. In certain example embodiments the mixture is heated to between about 35° C. to 45° C., thereby forming the second mixture.

Once the first mixture and the second mixture are formed, the two mixtures are combined under shear mixing conditions. For example, about 10-30% of the first (aqueous phase) mixture is combined under shear with about 70-90% of the second mixture, thereby producing a warmed liquid fill material. That is, the liquid fill material has the same composition as the semi-solid fill material composition described herein, but because the second mixture is heated, the fill is liquid, thereby allowing the incorporation of the fill into a softgel as described herein. In certain example embodiments, the liquid fill material includes about 10, 15, 20, 25, or 30% of the first mixture and about 70, 75, 80, 85, or 90% of the second mixture.

As those skilled in the art will appreciate, any high shear mixers known in the art can be used to achieve the high shear mixing described herein. Example high shear mixers include those supplied under the brands “TK Products Homomic Line Mill” or “Bematek” or “Greerco” or “Ross.” Example suppliers include IKA WORKS, Kady International, Charles Ross and Son Company, Silverson Machines, and Pulsar. A specific mixer that can be used in accordance with the methods and processes described herein is the Ross™ HSM-405SC-25 high shear mixer. In certain example embodiments, the rotor speed of the mixer is around 3,600 revolutions per minutes (rpm), such as around 1,400 rpm, 1,600 rpm, 1,800 rpm, 2,000 rpm, 2,200 rpm, 2,400 rpm, 2,600 rpm, 2,800 rpm, 3,000 rpm, 3,200 rpm, 3,400 rpm, 3,600 rpm, 3,800 rpm, 4,000 rpm, 4,200 rpm, 4,400 rpm, 4,600 rpm, 4,800 rpm, 5,000 rpm, 5,200 rpm, 5,400 rpm, 5,600 rpm or higher. In certain example embodiments, the rotor speed of the mixer is between 2,000 rpm to 6,000 rpm. In certain example embodiments, the rotor speed is between 3,000 rpm and 5,000 rpm. In certain example embodiments, the rotor speed is between 3,000 rpm and 4,000 rpm. With such high shear mixing, for example, hygroscopic emulsifiers can be mixed into the aqueous phase. Further, the high shear mixing combines the lipid phase with the aqueous phase to form the final fill material, in accordance with the methods and processes provided herein.

Once the liquid fill material is formed, the fill material can be included into a softgel using conventional means, such as described in U.S. Pat. Nos. 9,433,559, 9,638,464, and 8,621,764, each of which are hereby incorporated herein by reference in their entirety. For example, and as those skilled in the art will appreciate, soft film ribbons of gelatin are formed from a film-forming composition. That is, the film-forming composition used to prepare the softgels can be prepared according to formulations known to those skilled in the art. The film-forming composition can be any suitable composition for making softgels. The properties of the film-forming composition are determined, at least in part, by the cohesive strength of the constituent gelatin, expressed as “bloom”. In certain example embodiments, the gelatin film formulation includes 219.0 kg of gelatin 150 bloom, 110.0 kg of glycerin 99.5%, and 172.5 kg of purified water and 6.5 kg of caramel color. In a certain example embodiment, the gelatin film formulation includes between about 37% and about 41% 150 bloom gelatin, such as bloom bovine gelatin, between about 17% and about 21% glycerin and between about 25% and about 29% water.

In certain example embodiments, preparing the gelatin film includes pre-weighing all raw materials into clean containers, adding glycerin and purified water to a gelatin melter (which is set in an example embodiment to 176° F.), and then mixing the material until the material reaches 176° F. At that point, pre-weighed raw gelatin is added. A vacuum is then applied to allow the liquids to rise and saturate the gelatin. The vacuum is removed, the tank with the mixture is sealed with the vacuum. The mixture is then allowed to mix for about 30 minutes in/on a mixer/agitator, and the gelatin is de-aerated. The vacuum valve on the gelatin melter is left closed to seal the vacuum and the vacuum pump is turned off. The gelatin is then allowed to mix under sealed vacuum for 10 minutes at slow mixing speed, or until the temperature is between about 149° F. to about 158° F.

Once the gelatin mixture is prepared, the gelatin films are formed into ribbons by methods known to those skilled in the art, such as via a rotary die process. The ribbons are then fed through two mated die rolls of a gelatin encapsulation machine. For example, two gelatin ribbons are supplied to and between a pair of rotating and mated die rolls from the upper side of the die rolls, one from the right and one from the left, the die rolls being configured as described herein. The two mated die rolls are close to and confront each other. A softgel containing two half-capsule shells is formed by closing in the rotating die rolls. The liquid fill composition, such as that described herein, is then injected into the interior of the softgel with pressure to convert the otherwise flat softgel body into a swollen body of the softgel.

Once the softgels, including the fill compositions described herein, are formed the softgels can be advantageously dried using a variety of known methods. In certain example embodiments, the softgels can be dried as described in U.S. Pat. Nos. 9,638,464 and 8,621,764. For example, the drying method can include dividing a space into first, second and third zones and providing a first air handler unit for discharging air into the first zone. The first zone includes a first temperature sensor and a first humidity sensor that are both in communication with the first air handler unit. A second air handler unit is provided for discharging air into the second zone, the second zone including a second temperature sensor and a second humidity sensor that are both in communication with the second air handler unit. A third air handler unit is also provided for discharging air into the third zone. The third zone includes a third temperature sensor and a third humidity sensor that are both in communication with the third air handler unit.

The drying method can further include, for example, providing air to the first air handler unit at a first temperature and a first relative humidity, wherein the air within the first air handler unit is conditioned such that it has a second temperature and a second relative humidity. The conditioned air is blown from the first air handler unit into the first zone. Air is provided to the second air handler unit at the first temperature and the first relative humidity. The air within the second air handler unit is conditioned such that it has a third temperature and a third relative humidity, the conditioned air being blown from the second air handler unit into the second zone. The method can further include providing air to the third air handler unit at the first temperature and the first relative humidity, the air within the third air handler unit being conditioned such that it has a fourth temperature and a fourth relative humidity. The conditioned air is blown from the third air handler unit into the third zone. The method further includes, for example, providing a series of tumble dryers that extends from the first zone, through the second zone and into the third, and drying the gelatin capsules by moving the gelatin capsules through the tumble dryers from the first zone to the third zone.

In certain example embodiments, the second temperature is between about 50° F. and about 68° F. and the second relative humidity is between about 19% and about 23%. The third temperature is between about 72° F. and about 87° F. and the third relative humidity is between about 9% and about 14%. The fourth temperature is between about 68° F. and about 74° F. and the fourth relative humidity is between about 10% and about 15%. In certain example embodiments, the second temperature is between about 59° F. and about 61° F. and the second relative humidity is between about 20.5% and about 21.5%. The third temperature is between about 81° F. and about 83° F. and the third relative humidity is between about 10.5% and about 11.5%. The fourth temperature is between about 71° F. and about 73° F. and the fourth relative humidity is between about 12.5% and about 13.5%.

In certain example embodiments, the second temperature is about 60° F. and the second relative humidity is about 21%. The third temperature is about 82° F. and the third relative humidity is about 11%. The fourth temperature is about 72° F. and the fourth relative humidity is about 13%. In certain example embodiments, the air provided to the first, second and third air handler units has a first dew point. The air within the first air handler unit is conditioned such that it has a second dew point, the air within the second air handler unit is conditioned such that it has a third dew point, and the air within the third air handler unit is conditioned such that it has a fourth dew point. For example, the second dew point is between about 15° F. and about 30° F., the third dew point is between about 15° F. and about 23° F., and the fourth dew point is between about 15° F. and about 23° F. In certain example embodiments, the first air handler unit releases the conditioned air into the first zone at between about 3000 CFM and about 6000 CFM. The second air handler unit releases the conditioned air into the second zone at between about 2500 CFM and about 5000 CFM. The third air handler unit releases the conditioned air into the third zone at between about 1000 CFM and about 3000 CFM.

In certain example embodiments, the softgels can be dried to a hardness of eight newtons in about thirteen hours. In certain example embodiments, the first, second and third air handler units are positioned within the first, second and third zones, respectively. In certain example embodiments, the air provided to the first, second and third air handler units is provided by a HVAC unit that is positioned outside of the first, second and third zones.

Once dried, the softgels, including the fill material described herein, have a shelf life of several months to years. For example, the softgels, including the fill material described herein, may have a shelf life of about 2, 3, 4, 5, 6, 7, 8, 9, 10, 11, 12, 14, 16, 18, 20, 22, or 24 months. In certain example embodiments, the shelf life of the softgels, including the fill material described herein, may depend on the shelf life of the active ingredient included in the fill material. For example, if the active ingredient included in the fill material has a short shelf life, the softgel, including the active ingredient as part of the fill material, may have a shorter shelf life. In certain example embodiments, adding beeswax or other similar tough wax, as described herein, may prolong the shelf life of the softgel, such as by helping to maintain the shape and structural integrity of the softgel for a longer period of time as compared to softgels not including the beeswax. For example, including beeswax in the fill material described herein may extend shelf life of the softgel, as compared to a softgel including the fill material described herein but not including beeswax, by 3, 4, 5, 6, 7, 8, 9, 10, 11, 12, 14, 16, 18, 20, 22, or 24 months.

### EXAMPLES

The present invention is described in further detail in the following examples which are not in any way intended to limit the scope of the invention as claimed. The following examples are offered to illustrate, but not to limit the claimed invention.

### Example 1—Conventional Oil-Filled Softgels

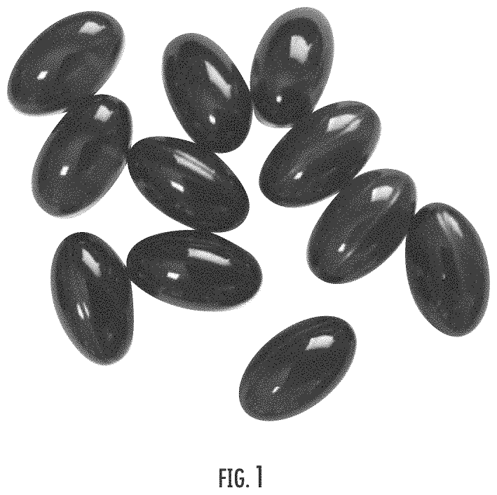

Softgels containing 99.8% fish oil were prepared and dried according to the methods described in U.S. Pat. Nos. 9,433,559, 9,638,464, and 8,621,764. Briefly, the softgel fill material included 99.8% fish oil, with the balance being rosemary extract, ascorbyl palmitate, ascorbic acid, soy lecithin, sunflower lecithin, and d-alpha tocopherol. As shown in FIG. 1, the resulting softgels have shells that are smooth and fully formed when conventionally dried.

### Example 2—Softgels Including Oil-In-Water Emulsion

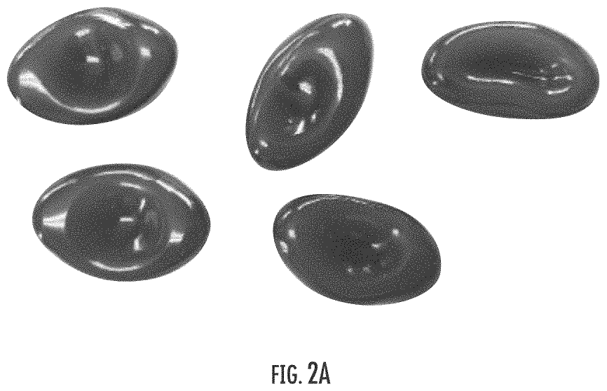

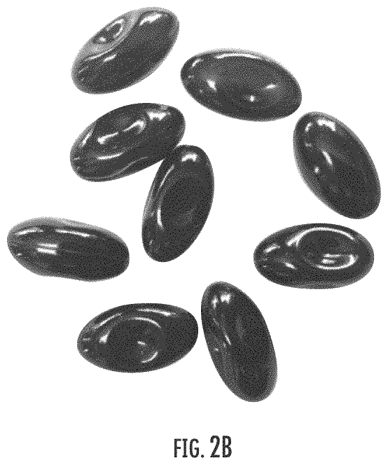

Softgels were produced using an oil-in-water (i.e., oil in polar solution include water/glycerin) emulsion. More particularly, for this example, the fill material included 28.7% water, 34% oil, and 4.7% of an emulsifier (gum Arabic, xanthan gum, guar gum), with the balance being xylitol, glycerin, citric acid, sorbic acid, coloring agent, natural flavorings, ascorbyl palmitate, and vegetable juice. The fill material was then used to fill softgels as described in U.S. Pat. No. 9,433,559. Before the softgels were dried, the softgel shells had a dimpled appearance and were not fully formed (FIG. 2A). The softgels were then dried according to U.S. Pat. Nos. 9,638,464 and 8,621,764. After drying, however, the softgels with the oil-in-water emulsion continued to have a wrinkled and dimpled appearance (FIG. 2B). The softgels were also overly soft, lacking the structural integrity of a conventional softgel. The softgels also lost their opacity, suggesting a broken emulsion in the fill, i.e., that water of the emulsion had migrated out of the fill material.

### Example 3—Softgels Including Waxy and Non-Waxy Components

In this example, softgels including the fill material described herein were prepared. Briefly, a first mixture (aqueous phase) was prepared using 50% water, 40% glycerin and 10% emulsifier (3% gum Arabic and 7% starch). The first mixture was then mixed at room temperature using a high shear Ross™ HSM-405SC-25 set at 100% speed until the mixture was uniform in appearance. The first mixture was then stored until needed.

To prepare the second mixture (oil phase), in a separate container CBD oil (in a paste) was mixed with coconut oil to form the first lipid component (i.e., the waxy oil component). Thereafter, 73.5% of the waxy oil was combined with 10% non-waxy (liquid) MCT oil (MCT 55/45, by Wilmar™), 11% TPGS, and 5.5% lecithin at a temperature of about 45° C. The second mixture was then mixed until uniform without high-shear mixing.

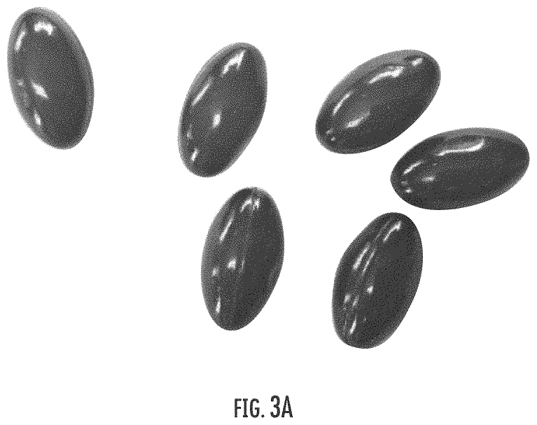

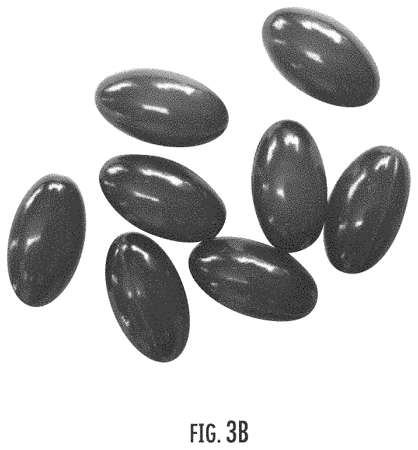

Thereafter, the first and second mixtures were combined and mixed with a high shear Ross™ HSM-405SC-25 set at 100%, thereby forming a liquid fill material for the softgels. Using the liquid (melted) fill material maintained at 35-45° C., softgels were then prepared as described in U.S. Pat. Nos. 9,433,559, 9,638,464, and 8,621,764. The softgels had a normal shape and were opaque before drying (FIG. 3A). The softgels were then cooled and dried according to U.S. Pat. Nos. 9,638,464, and 8,621,764. As shown in FIG. 3B, even though the softgels included 5.75% water in final fill material, the softgels having the fill material described in this example possessed a smooth appearance, were fully formed, and possessed structural integrity suitable for packaging and shipping. The fill material was also opaque after drying, indicating an intact water-in-oil emulsion in the fill material.

### Example 4—Softgels Lacking Waxy Lipid Component

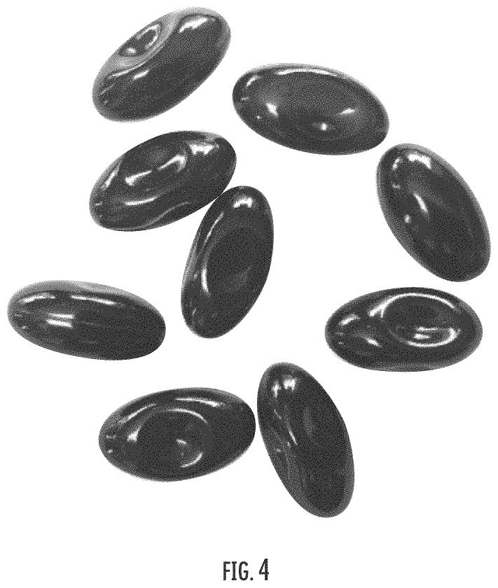

In this example, softgels including the fill material described herein were prepared according to the methods described in Example 3 (above), but without the waxy oil component. In other words, only the liquid non-waxy oil was used. Briefly, the fill material included 73.2% non-waxy CBD oil, 13.1% water, 9.2% glycerin, 0.2% emulsifier (gum Arabic, xanthan gum, and/or guar gum), 0.4% TPGS, and 3.9% lecithin. The softgels were then dried according to U.S. Pat. Nos. 9,638,464 and 8,621,764. The softgels are shown in FIG. 4. As shown, the softgels were soft (uncured) and deformed after drying. The fill material within the dried softgels was also clear, indicating that all the water and emulsifier had migrated from the fill material into the softgel wall and/or into the environment, breaking the emulsion.

### Example 5—Softgels Including High Ratio of Waxy to Non-Waxy Components

In this example, softgels including the fill material described herein were prepared. Briefly, a first mixture (aqueous phase) was prepared using 40% water, 40% glycerin, and 20% emulsifier (EmulTru™, from Cargil™). The first mixture was then mixed at room temperature using a high shear Ross™ HSM-405SC-25 set at 100% speed until the mixture was uniform in appearance. The first mixture was then stored until needed. The composition of the aqueous phase is shown in Table 1 below.

TABLE 1
Aqueous phase components of Example 5.

| Ingredients | % by Wt. |
|---|---|
| Water | 40% |
| Glycerin | 40% |
| EmulTru ™ (Emulsifier) | 20% |

To prepare the second mixture (oil phase), in a separate container CBD oil (in a paste) was mixed with coconut oil to form the first lipid component (i.e., the waxy oil component). Thereafter, 73.5% of the waxy oil was combined with 6% non-waxy (liquid) MCT oil (MCT 55/45, by Wilmar™), 15% TPGS, and 5.5% lecithin at a temperature of about 45° C. The second mixture was then mixed until uniform without high-shear mixing. The composition of the oil phase is show in Table 2 below.

TABLE 2
Oil (lipid) phase components of Example 5.

| Ingredients | % by Wt. |
|---|---|
| TPGS | 15% |
| Lecithin | 5.5% |
| MCT oil (non-waxy) | 6% |
| Coconut + CBD blend (total waxy) | 73.5% |
| Coconut oil (waxy) | 60.68% |
| CBD blend (waxy) | 12.82% |
| Ratio of waxy to non-waxy | 12.25:1 |

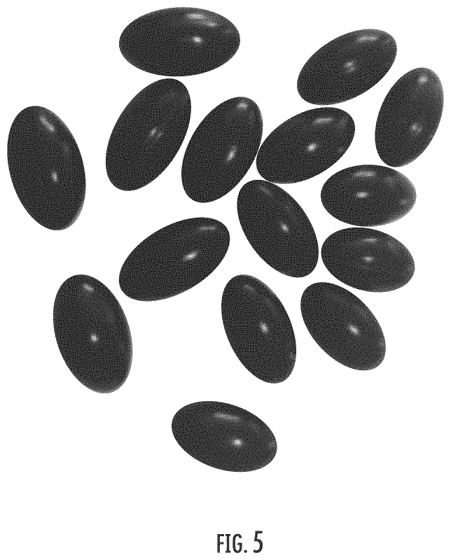

Thereafter, the first and second mixtures were combined and mixed with a high shear Ross™ HSM-405SC-25 set at 100%, thereby forming a liquid fill material for the softgels. Using the liquid (melted) fill material maintained at 35-45° C., softgels were then prepared as described in U.S. Pat. Nos. 9,433,559, 9,638,464, and 8,621,764. The softgels had a normal shape and were opaque before drying (FIG. 5). The softgels were then cooled and dried according to U.S. Pat. Nos. 9,638,464, and 8,621,764. As shown in FIG. 5, even though the softgels included 4.6% water in final fill material, the softgels having the fill material described in this example possessed a smooth appearance, were fully formed, and possessed structural integrity suitable for packaging and shipping, with a shelf life of at least 2 months. The fill material was also opaque after drying, indicating an intact water-in-oil emulsion in the fill material. The composition of the final fill material is shown in Table 3, below.

TABLE 3
Final softgel fill material of Example 5.

| Ingredients | % by Wt. |
|---|---|
| TPGS | 13.275% |
| Lecithin | 4.87% |
| MCT oil (non-waxy) | 5.31% |
| Coconut + CBD blend (waxy) | 65.05% |
| Coconut | 56.71 |
| CBD blend | 8.34 |
| Ratio of waxy to non-waxy | 12.25:1 |
| Water | 4.6% |
| Glycerin | 4.6% |
| EmulTru ™ (Emulsifier) | 2.3% |

### Example 6—Softgels Including High Ratio of Waxy to Non-Waxy Components and Beeswax

In this example, softgels including the fill material described herein were prepared, the fill material included a high ratio of waxy to non-waxy components along with beeswax. Briefly, a first mixture (aqueous phase) was prepared using 35% water, 35% glycerin, and 30% emulsifier (EmulTru™, from Cargil™). The first mixture was then mixed at room temperature using a high shear Ross™ HSM-405SC-25 set at 100% speed until the mixture was uniform in appearance. The first mixture was then stored until needed. The composition of the aqueous phase is shown in Table 4 below.

TABLE 4
Aqueous phase components of Example 5.

| Ingredients | % by Wt. |
|---|---|
| Water | 35% |
| Glycerin | 35% |
| EmulTru ™ (Emulsifier) | 30% |

To prepare the second mixture (oil phase), in a separate container CBD oil (in a paste) was mixed with coconut oil to form the first lipid component (i.e., the waxy oil component). Thereafter, 76.5% of the waxy oil component (coconut oil, CBD, and beeswax) was combined with 3% non-waxy (liquid) MCT oil (MCT 55/45, by Wilmar™), 15% TPGS, and 5.5% lecithin at a temperature of about 45° C. The second mixture was then mixed until uniform without high-shear mixing. The composition of the oil phase is show in Table 5 below.

TABLE 5
Oil (lipid) phase components of Example 6.

| Ingredients | % by Wt. |
|---|---|
| TPGS | 15% |
| Lecithin | 5.5% |
| MCT oil (non-waxy) | 3.00% |
| Beeswax (waxy) | 7.000% |
| Coconut + CBD blend (total waxy) | 69.5% |
| Coconut oil (waxy) | 59.44% |
| CBD blend (waxy) | 10.06% |
| Ratio of waxy to non-waxy | 25.5:1 |

Thereafter, the first and second mixtures were combined and mixed with a high shear Ross™ HSM-405SC-25 set at 100%, thereby forming a liquid fill material for the softgels. Using the liquid (melted) fill material maintained at 35-45° C., softgels were then prepared as described in U.S. Pat. Nos. 9,433,559, 9,638,464, and 8,621,764. The softgels had a normal shape and were opaque before drying (data not shown). The softgels were then cooled and dried according to U.S. Pat. Nos. 9,638,464, and 8,621,764. Notably, even though the softgels included 4.6% water in final fill material, the softgels having the fill material described in this example possessed a smooth appearance, were fully formed, and possessed structural integrity suitable for packaging and shipping. The fill material was also opaque after drying, indicating an intact water-in-oil emulsion in the fill material. The softgels remained fully formed and with suitable structural integrity, even at six months after production. The composition of the final fill material, including beeswax, is shown below in Table 6.

TABLE 6
Final softgel fill material of Example 6.

| Ingredients | % by Wt. |
|---|---|
| TPGS | 13.275% |
| Lecithin | 4.868% |
| MCT oil (non-waxy) | 2.655% |
| Beeswax | 6.195% |
| Coconut + CBD blend (waxy) | 61.507% |
| Coconut | 52.604 |
| CBD blend | 8.903 |
| Ratio of waxy to non-waxy | 25.5:1 |
| Water | 4.025% |
| Glycerin | 4.025% |
| EmulTru ™ (Emulsifier) | 3.450% |

### Example 7—Particle Size Evaluation

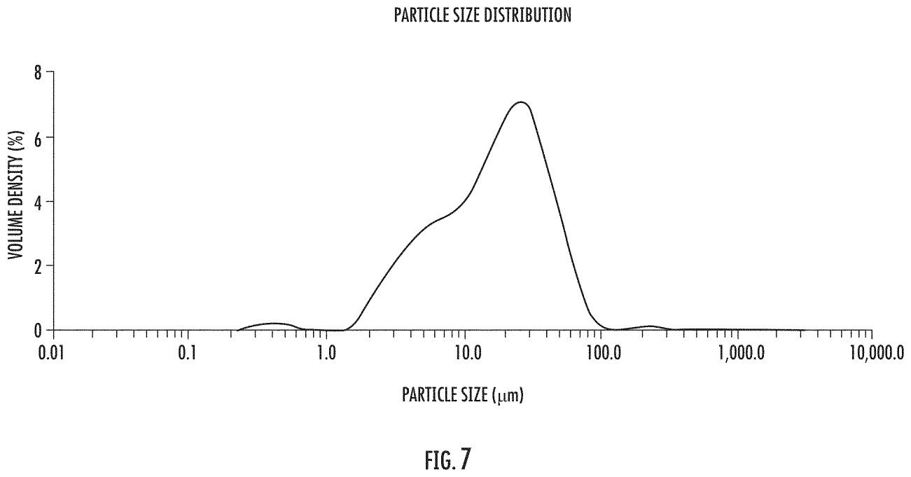

In this example, softgels were prepared according to Example 6. After cooling, a random sampling of five softgels were selected for particle size determination of the nonpolar (oil) component droplets within the softgels. Briefly, particle size analysis was conducted using a Malvern® MasterSizer 2000 LASER diffractor. As those skilled in the art will appreciate, the Malvern® MasterSizer LASER diffractor is considered an ensemble analyzer that calculates a volume distribution from the LASER (Light Amplification by Stimulated Emission of Radiation) diffraction pattern of a suspension of particles. The raw scatter data are then processed using the manufacturer's algorithm and presented on the basis of EQUIVALENT SPHERICAL DIAMETER. The results of the particle size analysis are shown below in Table 7. As can be seen, the particle size of the softgel fill material was highly consistent among the five softgel samples tested. A histogram of the particle size distribution for sample number 5 (see table 7) is shown in FIG. 7. The particle size histogram for each of samples 1-4 (data not shown) was nearly identical to that of sample five shown in FIG. 7.

TABLE 7
Particle size for softgel fill samples (μm)

| | Sample number | | | | | |
|---|---|---|---|---|---|---|
| | 1 | 2 | 3 | 4 | 5 | Average |
| D(10) | 3.94 | 3.91 | 3.9 | 3.87 | 3.9 | 3.904 |
| D(50) | 17.9 | 17.7 | 17.6 | 17.4 | 17.6 | 17.64 |
| D(90) | 46.2 | 45.8 | 45.2 | 44.1 | 45.3 | 45.32 |

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

I claim:

1. A softgel comprising a semisolid fill material, wherein the semisolid fill material comprises:
(a) a first lipid component and a second lipid component, wherein the first lipid component and the second lipid component comprise a total of at least 60% by weight of the fill material;
(b) d-α-tocopheryl polyethylene glycol succinate (“TPGS”) or a derivative thereof; and
(c) water and/or other polar solvent, wherein the total amount of the water and/or other polar solvent comprises at least 3% by weight or less of the fill material.

2. The softgel of claim 1, wherein the semisolid fill material comprises less than about 5% by weight of an emulsifier.

3. The softgel of claim 1, wherein the first lipid component is a waxy oil or mixture of waxy oils and wherein the second lipid component is a non-waxy oil or mixture of non-waxy oils.

4. The softgel of claim 3, wherein the waxy oil or mixture thereof and the non-waxy oil or mixture thereof are present in a ratio of at least 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1 or 30:1.

5. The softgel claim 1, wherein the semisolid fill material comprises at least 40% by weight coconut oil.

6. The softgel of claim 1, wherein the semisolid fill material comprises at least 4% beeswax.

7. The softgel of claim 1, wherein the semisolid fill material comprises a nonpolar active ingredient.

8. The softgel of claim 7, wherein the active ingredient is a cannabidiol (CBD), a fish oil, a flax seed oil, a black seed oil, or combination thereof.

9. The softgel of claim 1, wherein the semisolid fill material comprises about 4-6% by weight of the lecithin.

10. The softgel of claim 1, wherein the semisolid fill material comprises about 3-6% by weight of the glycerin.

11. The softgel of claim 1, wherein the semisolid fill material comprises at least 4-8% by weight of the water and/or other polar solvent.

12. The softgel of claim 1, wherein the first lipid component and the second lipid component comprise a total of at least 65% by weight of the fill material.

13. The softgel of claim 1, wherein the semisolid fill material comprises about 8-15% by weight of the TPGS or derivative thereof.

14. The softgel of claim 1, wherein the first and second lipid components are dispersed within the water and/or other polar solvent and wherein the first and second lipid components form droplets within the dispersion, the droplets having a median or average particle size of about 15 to 20 μm.

15. A semisolid softgel fill material, comprising:
(a) a waxy oil and a non-waxy oil, wherein the waxy oil and the non-waxy oil comprise at least 60% of the fill material and wherein the waxy oil and the non-waxy oil are present in a ratio of at least 10:1;
(b) a surfactant;
(c) one or more emulsifiers; and
(d) water and/or other polar solvent, wherein the total amount of the water and/or other polar solvent comprises about 10% or less of the fill material.

16. The semisolid softgel fill material of claim 15, wherein the emulsifier comprises about 5% or less of the semisolid softgel fill material.

17. The semisolid softgel fill material of claim 15, wherein the waxy oil and the non-waxy oil are present in the fill material at a ratio of at least 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1 or 30:1.

18. The semisolid softgel fill material claim 15, wherein the fill material comprises at least 4% by weight beeswax.

19. The semisolid softgel fill material of claim 1, wherein the active ingredient is a nonpolar active ingredient.

20. The semisolid softgel fill material of claim 1, wherein the semisolid fill material further comprises about 4-6% by weight lecithin, about 3-6% by weight glycerin, about 8-15% by weight d-α-tocopheryl polyethylene glycol succinate (“TPGS”), and wherein the waxy oil and the non-waxy oil comprise at least 65% by weight of the fill material.

21. The semisolid softgel fill material of claim 20, wherein the semisolid softgel fill material comprises droplets of the waxy and non-waxy oils, the droplets having a median or average diameter of about 15 to 20 μm.
